(19) United States
(12) Patent Application Publication
(10) Pub. No.: US 2026/0157685 A1
(43) Pub. Date: Jun. 11, 2026

(54) SYSTEM AND METHODS FOR PERFORMING A REMOTE HAIR ANALYSIS

(71) Applicant: Spider Medical Ltd., Nahariya (IL)

(72) Inventors: Zaher ANDRAUS, Ann Arbor, MI (US); Timothy LANE, Ypsilanti, MI (US); Yaer LIBERMAN, Victoria (AU)

(21) Appl. No.: 18/708,154

(22) PCT Filed: Nov. 8, 2022
(86) PCT No.: PCT/IL2022/051181

§ 371 Date: May 7, 2024

Related U.S. Application Data
(60) Provisional application No. 63/276,694, filed on Nov. 8, 2021.

(51) Int. Cl. A61B 5/00

(52) U.S. Cl. CPC A61B 5/448; A61B 5/0013; A61B 5/0077; A61B 5/7264

(57) ABSTRACT

A system for performing a remote hair analysis of the head of a hair restoration candidate, comprising one or more image-capturing devices (such as a smartphone a tablet, a digital camera or a LIDAR sensor) adapted to connect directly, or through a computer, to the internet, for capturing images or video segments of the head of the hair restoration candidate to be processed, and for transmitting the captured images or video segments to one or more central computing devices; a central computing device, configured with suitable hardware for running an operating software, which is configured to receive images or video segments of hair restoration candidates' heads, and to process and analyze perceptible hair parameters therein for inferring as to the candidates' eligibility for a hair restoration procedure.

### FIELD OF THE INVENTION

The present invention relates to the field of hair transplant. More specifically, the invention relates to a system and method for performing a remote hair analysis.

### BACKGROUND OF THE INVENTION

Patients who are candidates for hair restoration procedures are required to visit the hair restoration clinic prior to the surgery for a thorough inspection. This inspection is critical to determine potential patients' eligibility for the hair restoration procedure. One inspected parameter that affects a patient's eligibility is hair coverage of the patient's scalp, where patients with hair coverage higher than a predetermined threshold are defined as good candidates for the procedure, while patients with hair coverage lower than a predetermined threshold may be considered ineligible. Furthermore, a patient with a borderline recession or hair coverage condition requires further analysis by the clinic, to reach a recommendation, plan, or proposal as to a suitable hair restoration procedure.

Unfortunately, many candidates do not approach the hair restoration procedure due to the distance they need to travel (at least twice) to the hair restoration clinic, and moreover, due to the chance of finding they are ineligible for the procedure on their first travel there.

Currently available hair restoration systems use complex scanners for scanning and analyzing images of the candidate's head. However, these scanners are expensive and require visiting a hair restoration clinic.

It is therefore an object of the present invention to provide a system and method for performing remote hair analysis, for making the assessment of candidates for hair restoration procedures accessible to remote candidates.

It is another object of the present invention to provide a system and method for performing remote hair analysis, which does not require using complex scanners.

Other objects and advantages of the invention will become apparent as the description proceeds.

### SUMMARY OF THE INVENTION

A system for performing a remote hair analysis of the head of a hair restoration candidate, comprising:
a) one or more image-capturing devices (such as a smartphone a tablet, a digital camera or a LIDAR sensor) adapted to connect directly, or through a computer, to the internet, for capturing images or video segments of the head of the hair restoration candidate to be processed, and for transmitting the captured images or video segments to one or more central computing devices; and
b) one or more central computing devices, configured with suitable hardware for running an operating software, which is configured to receive images or video segments of hair restoration candidates' heads, and to process and analyze perceptible hair parameters therein for inferring as to the candidates' eligibility for a hair restoration procedure.

The one or more central computing devices may be configured to receive and analyze consecutive images or video segments of a candidate's head (which may be shaved or unshaved), and to identify one or more anchoring follicles within two or more consecutive images or video frames.

The one or more image-capturing devices may be adapted to perform a partial or complete analysis of the captured images or video segments.

The images or video segments may be captured by manually moving the image-capturing devices by the candidate or by another assisting person.

A method for performing a remote hair analysis of the head of a hair restoration candidate, comprising:
a) capturing, by one or more image-capturing devices adapted to connect directly, or through a computer, to the internet, images or video segments of the head of the hair restoration candidate to be processed;
b) transmitting the captured images or video segments to one or more central computing devices;
c) receiving images or video segments of hair restoration candidates' heads; and
d) processing and analyzing perceptible hair parameters, for inferring as to the candidates' eligibility for a hair restoration procedure.

The analysis comprises the classification of pixels as related to hair within a received image, or a single frame of a head scan video of the candidate's head, may be performed by:
a) receiving an input image frame of a shaved head as R, G, B pixels; and
b) classifying pixels that can be associated with human hair follicles.

The method may comprise the steps of:
a) converting the input image to grayscale;
b) identifying pixels which can be associated with hair; and
c) combining the classification results from the preceding step, based on logical functions or weighted sums, or others.

The analysis comprises the identification of individual hair follicles formed by groups of residing pixels identified as hair by:
a) mapping Pixels grid with “hair” and “scalp” and labeling classification results; and
b) Grouping pixel labels to identify full hair.

The method may comprise the steps of:
c) identifying full hair by linking the pixels identified as hair according to the previous section into groups, based on full adjacency; and
d) for each frame, estimating the number of follicles and their location.

The analysis may comprise the identification of hair follicles and the number of hairs within each hair follicle by:
a) receiving hair classification and one hair follicle with its bounding box; and
b) grouping pixel labels to identify full hair and detect the number of hair within the hair follicle.

The method may comprise the steps of:
a) for every bounding box, creating an orthogonal line that goes through the bounding box;
b) scanning pixels and counting the number of times they flip from H to S and vice versa in each classified image; and
c) Returning the maximal count from the preceding step.

The analysis may comprise the generation of a profile of each identified hair follicle by:
a) receiving each Hair follicle represented by a predetermined number of pixels; and
b) characterizing each hair follicle according to its orientation and the number of hairs within the follicle.

The method may further comprise the step of analyzing a series of consecutive images, or a video clip as a series of consecutive frames, where anchoring follicles identified within two or more consecutive frames, are used as references to calculate the displacement of the anchor follicles between the consecutive frames.

The analysis may comprise the steps of:
a) receiving video segments of a shaved head; and
b) identifying and characterizing the hair follicles on each scanned segment of the shaved head.

The method may comprise the steps of:
a) for each frame, identifying and characterizing hair follicles in the frame and in its subsequent frame;
b) Finding the intersection group of follicles, to avoid excessive counting; and
c) Identifying follicles in the intersection group by estimating, for every follicle found in a frame, its location in the subsequent frame,
   where follicles without matches in consecutive frames are assumed to be new, and are added up to the general follicles count.

The analysis may comprise the identification and characterization of hair follicles on an unshaved head by:
a) cropping the raw head image to exclude objects and background and to leave only the desired head section; and
b) Classifying pixels that can be associated with a cropped human head.

Classification may be done by using a classifier based on the Convolutional Neural Network model, which was trained using deep learning, to identify objects on an image and segment the image into different classes.

Classification may be done by applying grabcuts algorithm for image segmentation, based on graph cuts with oval approximation.

The analysis may comprise the steps of:
a) receiving video segments of a shaved head; and
b) identifying and characterizing the hair follicles on each scanned segment of the shaved head.

The analysis may comprise the steps of:
a) receiving frames of video segments of a cropped head;
b) analyzing the frames and identifying pixels related to hair and other pixels, related to the scalp; and
c) classify pixels as related to hair or the scalp.

The method may comprise the step of determining the change in color of every pixel, compared to its neighborhood by:
a) applying Gaussian blur on the color image in an RGB color plane;
b) creating a new image of the distance from the original to the blurred image to, find localized changes;
c) applying a second Gaussian blur to the new image to find areas with substantial change; and
d) classifying pixels with rapid changes with respect to their neighboring pixels as hairs.

The method may comprise the step of analyzing the luminosity and saturation of the images in the HLS color plane by:
c) converting the image from RGB to HLS color plane;
d) estimating the geometric mean of L and S components for the neighborhood of every pixel;
e) applying Mean Threshold to estimate the differences in lighting for each neighborhood;
f) removing outliers according to a predetermined threshold; and
g) scaling inliers to scale from “probably hair” (dark) to “probably skin” (light).

The analysis may comprise calculating the estimated hair coverage by:
a) receiving frames of video segments and cropping the head from each frame by:
creating a Semantic Segmentation Mask;
creating a Grabcuts Mask;
joining the masks to create a final cropping mask;
removing imperfections from the final cropping mask using morphological closing and opening;
b) classifying each pixel as scalp or hair by:
creating a Color Neighboring Classification;
creating a Luminosity and Saturation Classification;
combining the classifications into a final score;
determining a threshold for classifying pixels having a score below the threshold as related to scalp and pixels having a score above the threshold as related to hair;
c) calculating the final hair coverage percentage by:
applying the threshold on the final score image and calculating the percentage of the above-threshold score pixels being related to hair, with respect to the below-score pixels being related to skin;
counting the number of pixels classified as hair and dividing the counted number by the total number of pixels in the cropped image.

The method may further comprise the step of determining the candidate's eligibility for a hair restoration procedure according to his final hair coverage percentage.

A system for performing a remote hair analysis of the head of hair restoration candidate, comprising:
a) one or more image capturing devices adapted to connect directly, or through a computer, to the internet, for capturing images or video segments of the head of the hair restoration candidate to be processed, and for transmitting the captured images or video segments to one or more central computing devices; and
b) one or more central computing devices, configured with suitable hardware for running an operating software, which is configured to receive images or video segments of hair restoration candidates heads, and to process and analyze perceptible hair parameters therein for inferring as to the candidates' eligibility for a hair restoration procedure.

The one or more central computing devices may be configured to receive and analyze images a candidate's head, and to identify one or more follicles, and for each, one or more property like angle or number of hair, or to receive and analyze consecutive images or video of a candidate's head, and to identify a total collection of follicles and their hair on the candidate's head.

The one or more central computing devices may be configured to receive and analyze consecutive images or video of a candidate's head, and to identify one or more anchoring follicles within two or more consecutive images or video frames.

A total coverage in shaved head may be calculated from the total number of follicles or hair identified, and based on a total number of expected follicles or hairs in adults.

A total coverage in unshaved head may be calculated based on identifying the portion of the image that belongs to the head, classifying pixels that belong to the scalp versus the hair, and calculating the percentage of hair coverage accordingly.

A calibration method may be used to determine the equivalent value of each pixel in the image.

The calibration method may be performed by a device, such as a LIDAR device, to estimate the distance to the head.

The calibration method may be attaching a layout (such as a surface with millimetric marking.) with known distances on the head at the time of taking the pictures.

Measurements of the hair follicles such as width, density, or HMI, may be determined based on the pixels classified as hair, and the calibration metric per pixel.

A magnification device may be used in conjunction with the capturing device, in order to improve the resolution and clarity.

### BRIEF DESCRIPTION OF THE DRAWINGS

The above and other characteristics and advantages of the invention will be better understood through the following illustrative and non-limitative detailed description of embodiments thereof, with reference to the appended drawings, wherein:

FIG. 1 illustrates a system diagram of a system for performing a remote hair analysis, according to an embodiment of the present invention;

FIGS. 2A-2B show input and output images of a candidate's shaved head along hair analysis process thereof performed by the system of FIG. 1, according to an embodiment of the present invention;

FIG. 3 illustrates an exemplary analysis process of a series of consecutive images of candidates' shaved heads, according to an embodiment of the present invention;

FIGS. 4A-4C show images of a candidate's un-shaved head along the hair analysis process thereof, according to an embodiment of the present invention; and

FIG. 5 illustrates exemplary analysis processes of candidates' un-shaved heads, according to an embodiment of the present invention;

FIG. 6 is a schematic flowchart of the process of calculating the Eligibility of each candidate;

FIG. 7A shows an image frame of an unshaved scalp close-up with a millimetric layout attached to the scalp, with a known square dimension, as shown in FIG. 7A.

FIG. 7B shows classifying pixels using thresholding on the gradient results and blurring/smoothing (average over a window);

FIG. 7C illustrates performing the Hough line transform to detect vertical lines;

FIG. 7D shows collecting all the line's heights and taking the median value as the height of the slice, as shown in FIG. 7D.

FIG. 7E illustrates classify pixels to black lines vs white background using dynamic thresholding;

FIG. 7F illustrates fitting squares into the white areas and locating the square vertexes;

FIG. 7G. illustrates ordering each square's vertexes counter-clockwise, to find horizontal and vertical angles for each edge;

FIG. 7H illustrates using the rotation angle to rotate the image so that the vertical lines form 90° with the y-axis;

FIG. 8 shows a magnifier attached to the candidate's smartphone, for increasing the resolution;

FIG. 9 illustrates using the angle of each image from Step 2, obtain the average number of pixels making up its width along each hair.

### DETAILED DESCRIPTION OF EMBODIMENTS OF THE INVENTION

The present invention relates to a system that is adapted to receive and process images (i.e., photos and/or video streams) of hair restoration candidates' heads for determining their eligibility for hair restoration procedures.

The proposed system utilizes a set of hair analysis algorithms, for processing the received images to detect and analyze perceptible hair parameters therein (e.g., hair coverage), by which a candidate's eligibility for the hair restoration procedure is determined, where the analysis is performed in such a manner that does not require to know and/or to control the exact coordinates in space of the capturing device(s), thereby facilitating the use of basic capturing devices (e.g., common cameras connected to a mobile/desktop computers or a mobile device), that can be operated anywhere (e.g., at the comfort of candidates' homes) without requiring advanced photography capabilities or complex high-end equipment.

Generally, software modules include routines, programs, components, data structures, algorithms, and other types of structures that perform particular tasks or implement particular abstract data types. Moreover, those skilled in the art will appreciate that the invention may be practiced with other computer system configurations, including mobile devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

Embodiments of the invention may be implemented as a computer process (method), a computing system, or as an article of manufacture, such as a computer program product or computer-readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Therefore, unless otherwise indicated, the functions described herein may be performed by executable code and instructions stored in computer-readable medium and running on one or more processor-based systems. However, state machines, and/or hardwired electronic circuits can also be utilized. Further, with respect to the example processes described herein, not all the process states need to be reached, nor do the states have to be performed in the illustrated order. Further, certain process states that are illustrated as being serially performed can be performed in parallel. Similarly, while certain examples may refer to a central computer or a server, other computer or electronic systems can be used as well, such as, without limitation, a personal computer (PC), tablet, an interactive television, a smartphone (e.g., with an operating system and on which a user can install applications) and so on.

In the following detailed description, references are made to several embodiments of the present invention, examples of which are illustrated in the accompanying figures. The figures depict embodiments of the present invention for purposes of illustration only. One skilled in the art will readily recognize from the following description that the described embodiments may be combined, alternative embodiments may be utilized, and other changes may be made without departing from the spirit or scope of the present invention described herein.

The terms “for example”, “e.g.,”, and “for instance” as used herein, are intended to be used to introduce non-limiting examples. While certain references are made to certain example system components or services, other components and services can be used and/or the example components can be combined into fewer components and/or divided into additional components.

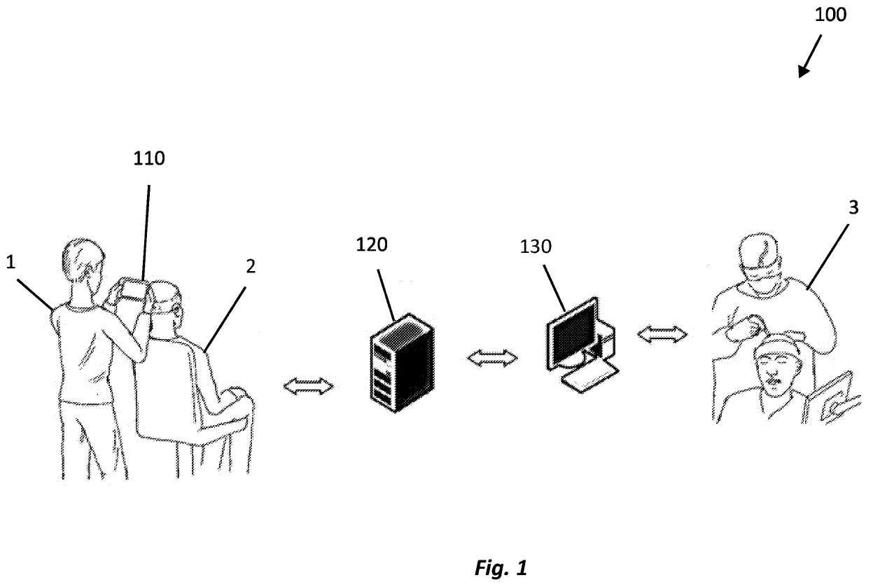

FIG. 1 illustrates a system diagram of a system 100 for performing a remote hair analysis, according to an embodiment of the present invention. System 100 comprises an image-capturing device 110, being operated by an assisting person 1 who aims and captures images of the head area of a hair restoration candidate 2. The capturing device may be any suitable handheld device (e.g., a smartphone, a tablet, a digital camera, etc.) which is adapted to connect to the internet directly or through a nearby computer, and the internet, to a central computing device (such as a server or a computational cloud) 120.

It should be understood by one skilled in the art that candidate 2 may also be guided by system 100, for capturing images without requiring the support of an assisting person 1. Furthermore, two or more capturing devices 110 may be used for providing enhanced scanning of a candidate's head.

Central computing device 120 (i.e., which may be alternatively realized as a distributed computer) runs a computer program (also referred herein as a “server program”) which is configured to run the hair analysis algorithms and to summarize the information and conclusions with respect to corresponding images received from capturing device 110, and to generate and submit corresponding reports thereof (e.g., eligibility report submitted to the candidate), while capturing device 110 (i.e., or a nearby computer connected thereto) runs a computer application (also referred herein as a “client program”), wherein the client program operates in conjunction with the server program, for receiving operational guidance associated to image capturing of the head of candidate 2 (also referred herein as a “head scan”), and/or transmittal of captured images to central computing device 120, or to perform partial or complete head scan analysis and transmitting the results thereof to central computing device 120.

The abovementioned server program of central computing device 120, may also operate with a web server program, while some capturing devices 110 (i.e., computers connected thereto) communicate with central computing device 120 through a corresponding web interface.

The concluded raw and analyzed data may be stored by central computing device 120, and/or submitted to the candidate (i.e., through the application or web interface operated by capturing device 110 or a computer connected thereto), and/or to hair restoration personnel (e.g., surgeon 3), by their internet-connected computer 130 or another computing device, through which surgeon 3 may provide final eligibility conclusions or further guidance for the head scan of candidate 2.

Now referring to several exemplary scenarios for performing the remote hair analysis process:

In one exemplary scenario, one or more photographs or video segments are obtained as part of the head scan using capturing device 110, and being locally analyzed on the same device using a hair analysis algorithm, such as the algorithms described below. In a second exemplary scenario, the photographs or video segments are transmitted over the internet to be processed via a hair analysis algorithm on a central computing device 120. In a third exemplary scenario, the algorithm is performed in part locally on capturing device 110 and in part remotely on central computing device 120.

Furthermore, the three scenarios above may be performed interactively, with the participation of surgeon 3, who may provide various inputs, such as providing guidance to the candidate (e.g., regarding the head area to be scanned), configuring or modifying analysis parameters, etc. Surgeon 3 may use computer 130 or any other computing device for interacting with the server program run by central computing device 120 and with the instant candidate there through. Further authorized users may also participate and contribute inputs to the analysis process such as the candidate's hair stylist (such as the required hairstyle or coverage after completing the restoration procedure).

Moreover, system 100 may also employ non-optical sensors such as Light Detection and Ranging (LIDAR) sensors, which can help with proximity (distance) estimation, and find more information related to the candidate's hair and hair deployment on the candidate's head. Knowing the distance allows for accurately estimating the pixel size.

System 100 is configured to facilitate two or more hair analysis processes, according to the circumstances. One exemplary process is for analyzing hair follicles on a candidate's shaved head, and another exemplary process is for analyzing hair follicles on a non-shaved head. Both exemplary processes are explained hereinafter.

### Identification and Characterization of Hair Follicles on Shaved Head

Shaved hair analysis enables identifying and characterizing discrete hair follicles since the hairs do not shade on each other, thereby enabling a more precise conclusion as to the hair coverage over the candidate's head, and as to the potential head sections, from which hair follicles can be taken for transplantation in desired hairless areas. Hence, when possible, hair analysis of shaved head is preferable for concluding as to the candidate's eligibility for hair restoration, as well as relevant hair restoration procedures for the instant candidate.

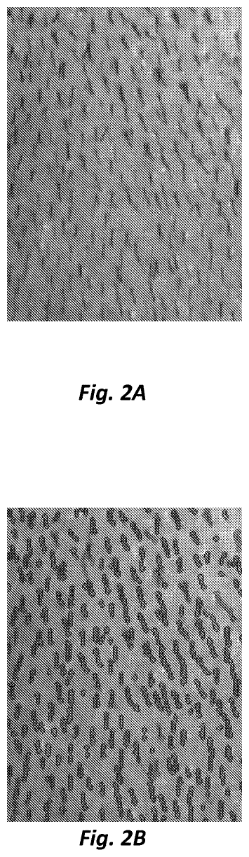

FIGS. 2A-2B illustrate exemplary analysis input and output images of a shaved head hair analysis process performed by system 100, according to an embodiment of the present invention. FIG. 2A is an input image and FIG. 2B is an output image in which the analysis results layer is displayed on top of the image of FIG. 2A. The analysis is performed by utilizing algorithms and analysis routines, as explained with reference to the following exemplary algorithms and analysis routines:

Analysis Routine 1—Classification of Pixels as Related to Hair within a Received Image, or a Single Frame of a Head Scan Video (Also Referred to Herein as “Hair Frame”) of the Candidate's Head
Input: An image frame of a Shaved Head given as Img(xₚ, yₚ)=(R_{x,y}, G_{x,y}, B_{x,y}) for pixels (xₚ, yₚ) on GRID(X, Y)
Output: (FIG. 2B) Classify pixels that can be associated with human hair follicle (as opposed to a scalp): C(Img(xₚ, yₚ)))∈{H, S}

### Exemplary Implementation:

1. Convert the image to Grayscale [1], i.e. build GS(Img(xₚ, yₚ)).
2. Identify pixels which can be associated with hair, using one or more classification methods, such as adaptive mean threshold [2], adaptive Gaussian mean threshold [2], or others, of the greyscale image. Each classification method will label a pixel C(GS(Img(xₚ,yₚ)))∈{H, S}
3. Combining the classification results from step 2 based on logical functions (e.g., OR/AND logic function), weighted sums, or others. The result can be combined uniformly for all pixels given one method, or used differently across the image. This analysis routine may also be slightly modified for performing the classification by RGB coordinates.

### Analysis Routine 2—Identifying Individual Hair (Also Referred to Herein as “Full Hair” or “HF”) Follicles Formed by Groups of Residing Pixels Identified as Hair by Routine 1

Input: Mapped Pixels grid with “hair” and “scalp” labeling: C(Img)
Output: Group pixel labels to identify full hair: HF(Img)={hf₁, hf₂, . . . |hfᵢ⊆Img}

### Exemplary Implementation:

1. Identifying full hair is done by linking the pixels identified as hair according to the previous section into groups based on full adjacency. Pixel (i, j) is fully adjacent to all pixels given by: (i+Δᵢ, j+Δⱼ) s.t. Δᵢ, Δᵢ∈{−1,0,1}, and Δᵢ and Δⱼ are not both 0, and i+Δᵢ≥0 and j+Δⱼ≥0 (eight or fewer pixels).
2. Based on that, an estimation of the number of follicles and their location is given per frame.
   Analysis Routine 3—Identify Hair Follicles and the Number of Hairs within Each Hair Follicle
Input: The hair classification C(Img), and one hair follicle hf∈HF(Img) with its bounding box (a 2D rectangle in which the hair length and orientation are confined) defined by pₗₒ, pₕᵢ and angle θ.
Output: Number of hair within hf

### Exemplary Implementation:

1. For every p_{L}∈(pₗₒ, pₕᵢ)—
   a. Create an orthogonal line that goes through p_{L}.

$$For θ < \frac{π}{2} : {L}_{o} = L ( ( Stretch ( {p}_{L} \cdot {θ}_{o} , \frac{D}{2} ) , {p}_{L} ) , ( {p}_{L} , Stretch ( {p}_{L} \cdot {θ}_{o} , \frac{D}{2} ) )$$

$$with {θ}_{o} = θ + \frac{π}{2} .$$

The formula is adjusted when

$$θ > \frac{π}{2} , with {θ}_{o} = θ - \frac{π}{2}$$

and adjusting the coordinates of Lₒ.
b. Scan Lₒ pixels and count the number of times they flip from H to S and vice versa in C(Img).
2. Return the maximal count from the above scan.

Step 1 may also be done on a single line in the center, instead of multiple lines.

### Analysis Routine 4—Generating a Profile of Each Identified Hair Follicle

Input: Hair follicle hf∈HF(Img) with k pixels
Output: Characteristics for hf including orientation (angle) and number of hairs within the follicle

### Exemplary Implementation for Said Characters:

1. Calculate the midpoint pixel:

$$( Round ( \frac{{\sum }_{p ∈ h f} {x}_{p}}{k} ) , Round ( \frac{{\sum }_{p ∈ h f} {y}_{p}}{k} ) )$$

2. Endpoint pixels: pₗₒ, pₕᵢ∈hf such that distance(pₗₒ, pₕᵢ) is maximal and yₚ_{ₗₒ}<yₚ_{ₕᵢ} (the definition of the order is immaterial, and so is the choice of using Y access for ordering instead of X)
3. Length: D=distance(p₁,p₂)
4. Angle: θ=θ(pₗₒ, pₕᵢ)
5. Number of follicles identified by Routine 3 with the above parameters.

Other methods to calculate the orientation and the number of hair within follicles are possible.

### Algorithm 1—Analyzing a Single Image or a Single Frame of a Video Clip of a Person's Head

### Definitions

A pixel and point, used interchangeably, p is defined by the pair (xₚ, yₚ) which corresponds to its coordinates, or location on a two-dimensional grid of width X and height Y called GRID(X, Y), such that 0≤xₚ<X and 0≤yₚ<Y

Given two points (pixels) p₁, p₂ then

$$distance ( {p}_{1} , {p}_{2} ) = \sqrt{{( {x}_{{p}_{1}} - {x}_{{p}_{2}} )}^{2} + {( {y}_{{p}_{1}} - {y}_{{p}_{2}} )}^{2}}$$

Given two points (pixels) pₗₒ, pₕᵢ such that yₚ_{ₗₒ}<yₚ_{ₕᵢ}, then:

$$θ ( {p}_{lo ,} {p}_{hi} ) = \{ \begin{matrix}{sin}^{- 1} ( \frac{distance ( {p}_{lo ,} {p}_{hi} )}{{y}_{{p}_{hi}} - {y}_{{p}_{hi}}} ) when {x}_{{p}_{hi}} \geq {x}_{{p}_{lo}} \\ π - {sin}^{- 1} ( \frac{distance ( {p}_{lo} , {p}_{hi} )}{{y}_{{p}_{hi}} - {y}_{{p}_{hi}}} ) otherwise\end{matrix}$$

Correspondingly, Stretch(pₗₒ, θ(pₗₒ, pₕᵢ), distance(pₗₒ, pₕᵢ))=pₕᵢ

Given two points (pixels) pₗₒ, pₕᵢ such that yₚ_{ₗₒ}<yₚ_{ₕᵢ}, then L(pₗₒ, pₕᵢ)⊆GRIX(X, Y) is a set of pixels p such that for each:
Input: Image frame with dimensions I and J of a Shaved Head given as Img(xₚ, yₚ)=(R_{x,y}, G_{x,y}, B_{x,y}) for pixels (xₚ, yₚ) on GRID(X, Y)
Output: Identifying and characterizing (orientation, number of hair) the hair follicles in the given frame.

### Exemplary Implementation:

1. Optionally—Pre-process Img to normalize some effects such as lighting effects, scene effects, and others (e.g., Empirical Line Calibration [3]). This can also be done on the entire set of frames (in Algorithm 2) to normalize the calibration.
2. Call Routine 1 for Img to classify/label pixels as hair versus scalp C(Img))
3. Call Routine 2 for C(Img)) to identify hair follicles HF(Img)
4. Call Routine 4 for every hair follicle hf∈HF(Img) to calculate its characteristics.

Other characteristics beyond orientation and number of hair, such as hair profile/curvature, angle, thickness, distribution, and others are possible. Furthermore, other data can be used beyond optical images, such as LIDAR info, to help map the spatial location of hair on the head.

Applying Algorihm1 on a given image (or video frame) results in identifying single hair follicles (blue) and double follicles (pink), as shown in FIG. 2A (pre-analysis) and FIG. 2B (post analysis).

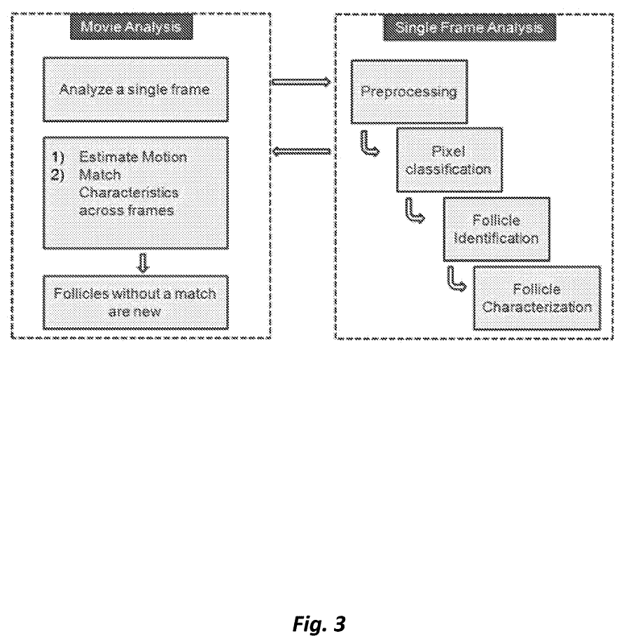

In order to obtain a more precise analysis of a candidate's head, a series of consecutive images, or a video clip is analyzed by system 100 and a series of consecutive frames is analyzed by algorithm 2 as illustrated in FIG. 3, and described hereinafter, which utilizes the abovementioned algorithm 1 for analyzing individual frames at a predetermined distance in time between the frames, where anchor follicles (i.e., individual follicles and groups thereof that are identified within two or more consecutive frames) are used as references to calculate the displacement of the anchor follicles between the consecutive frames, thereby parameters related to location, angle and length can be reaffirmed and be determined with higher accuracy, without requiring knowledge, calibration, or control (i.e., guiding candidate 2 or assistant person 1 to) of the exact position and orientation of capturing device 110.

### Algorithm 2

Input: Video (or series of frames/samples) of a Shaved Head, given by Img₁, . . . , Imgₙ
Output: Identifying (and Counting), and Characterizing the hair follicles on the scanned segment of the head

### Exemplary Implementation:

1. For each frame k=1, 2, . . . apply Algorithm 1 on Img and Imgₖ₊₁ to Identify and Characterize hair follicles.
2. Find the intersection group of follicles to avoid excessive counting:
a. Identification of follicles in the intersection group is done by estimation for every follicle found in frame Imgₖ, by estimating its location (via Mid-Point) in frame Imgₖ₊₁ using a number of possible methods. Examples:
i. Comparing sample areas across the two frames to estimate the motion.
ii. Comparing identified follicles across the frames to estimate the distance, then confirming by scoring comparisons of follicles based on the data for each (Mid-Point, Angle, Length)

Follicles without matches in consecutive frames are assumed to be new, and hence add up to the general follicles count.

This ongoing process enables the tracking of follicles along the video clip, and determines the total number of hair follicles while avoiding duplicate counting.

### Identification and Characterization of Hair Follicles on Unshaved Head

Some of the potential hair restoration candidates may not wish to shave their heads, particularly in favor of an initial examination, at the end of which they may not be found eligible for the hair restoration procedure. For such a case, system 100 is configured to perform a less accurate hair analysis process based on more general parameters such as hair coverage percentage of head sections which are prone to hair loss, and estimated hair characteristics (e.g., thickness).

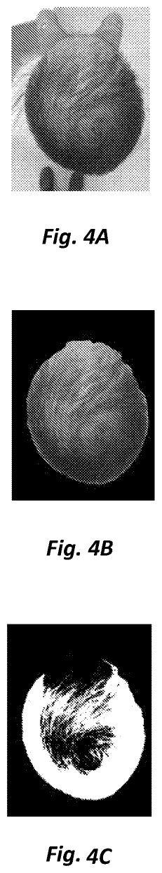

FIGS. 4A-4C illustrate the analysis stages of an un-shaved head as explained herein below.

The first step is cropping the raw head image (FIG. 4A) to exclude objects and background and to leave only the desired head section (FIG. 4B). This is performed by routine 5:

### Analysis Routine 5—Cropping the Received Image to Include Only the Head Frame to be Analyzed

Input: Image frame with dimensions I and J of an Un-Shaved Head given as Img(xₚ, yₚ)=(R_{x,y}, G_{x,y}, B_{x,y}) for pixels (xₚ, yₚ) on GRID(X, Y)
Output: Classify pixels that can be associated with (cropped) human head C(Img(xₚ,yₚ)))∈{0,1}

### Exemplary Implementation:

Apply Semantic Segmentation [4]—a Classifier based on the Convolutional Neural Network model which was trained using deep learning to identify objects on an image and segment the image into different classes.

### Another Exemplar Implementation:

Apply grabcuts algorithm [5]—an image segmentation method based on graph cuts (a semiautomatic segmentation technique that you can use to segment an image into foreground and background elements.) with oval approximation.

The second step is analyzing the cropped head image (FIG. 4B) to designate hair vs. scalp pixels and generate a mapping frame thereof (FIG. 4C). This is performed by routines 6 or 7:

Analysis Routine 6—Analyzing the Cropped Head Frame to Identify Pixels Related to a Hair Vs. Pixels Related to the Scalp
Input: The cropped head CH(Img)
Output: Classify pixels as hair or scalp: CNC(CH(Img))∈{H, S}

### Exemplary Implementation:

Determine the change in color of every pixel in comparison to its neighborhood. This is achieved by applying Gaussian blur [7] on the color image in the RGB color plane, creating a new image of the distance from the original to the blurred image to find localized changes and applying a second Gaussian blur to the new image to find areas with lots of change. Empirical results show that rapid changes between a pixel and its neighbors indicate hairs.

Analysis Routine 7—Analyzing the Cropped Head Frame to Identify Pixels Related to a Hair Vs. Pixels Related to the Scalp
Input: The cropped head CH(Img)
Output: Classify pixels as hair or scalp: LSC(CH(Img))∈{H, S}

### Exemplary Implementation:

The algorithm relies on analyzing the luminosity (luminance deals with the brightness of a certain color in the image) and saturation (saturation deals with the power or the saturation of a specific color) of the images in the HLS (HLS color model is a color model that defines colors by the three parameters hue (H), lightness (L), and saturation(S)) color plane. Empirical study shows a positive correlation between high saturation and skin classification, conditional to a saturation value. Therefore, the method includes converting the image from RGB to HLS color plane, estimating the geometric mean of L and S component for every pixel neighborhood and applying Mean Threshold to estimate the differences in lighting for each neighborhood, removing outliers according to the threshold, and scaling inliers to scale from “probably hair” (dark) to “probably skin” (light).

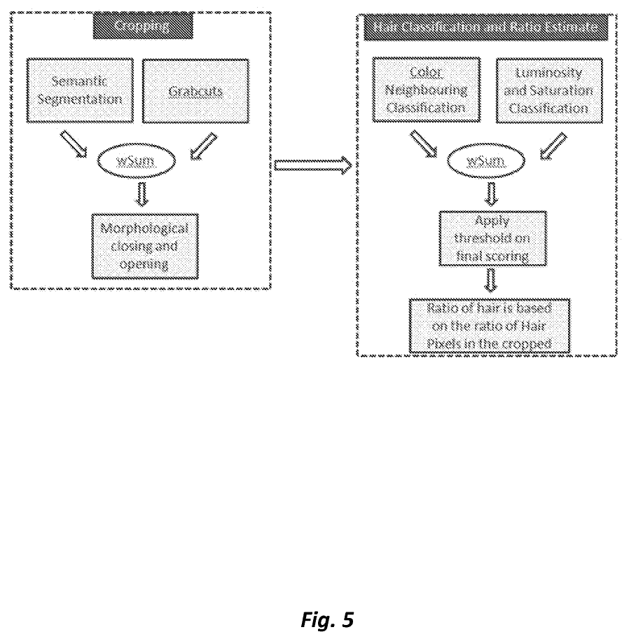

The resulting contrast mapping shown in FIG. 4C, is now analyzed to estimate the hair pixels (represented in white color in contrast to scalp pixels represented in black color) coverage over the head frame. This is performed by algorithm 3 described in FIG. 5 and herein below.

### Algorithm 3—Calculating the Estimated Hair Coverage

Input: Image frame with dimensions I and J of an Un-Shaved Head given as Img(xₚ, yₚ)=(R_{x,y}, G_{x,y}, B_{x,y}) for pixels (xₚ, yₚ) on GRID(X,Y)
Output: Estimating the percentage of hair over the head frame

### Exemplar Implementation:

1. Crop the head.
   a. Apply Routine 5 to create Semantic Segmentation Mask SSM(Img)
   b. Apply Routine 5 to create Grabcuts Mask GCM(Img)
   c. Apply them in sequence, for example, to GCM(SSM(Img))
   d. Given said masks, they are joined into creating a final cropping mask, for example, by conjoining the masks (a pixel is classified as head if and only if it is classified as head in all methods).
   e. Morphological closing and opening [6] are used to remove some small imperfections produced by either of the methods, or of the combined mask.
2. Given the final cropped head CH(Img), each pixel is classified as skin or hair as follows:
   a. Apply Routine 6 to create a Color Neighboring Classification CNC(CH(Img))
   b. Apply Routine 7 to create a Luminosity and Saturation Classification LSC(CH(Img))
   c. The different methods realized by routines 6 and 7 may be combined (or else, a single method is used) into a final score using, for example, weighted average, and rescaled to normalized classification results for the [0,255] segment. Thus, a threshold at 128 is set to decide to classify between the skin (lower score) and hair (higher score).
   d. Calculate the final hair percentage by applying a threshold on the final score image and calculating the percentage of the above-score pixels (hair) versus the below-score pixels (skin).

### Eligibility Calculation

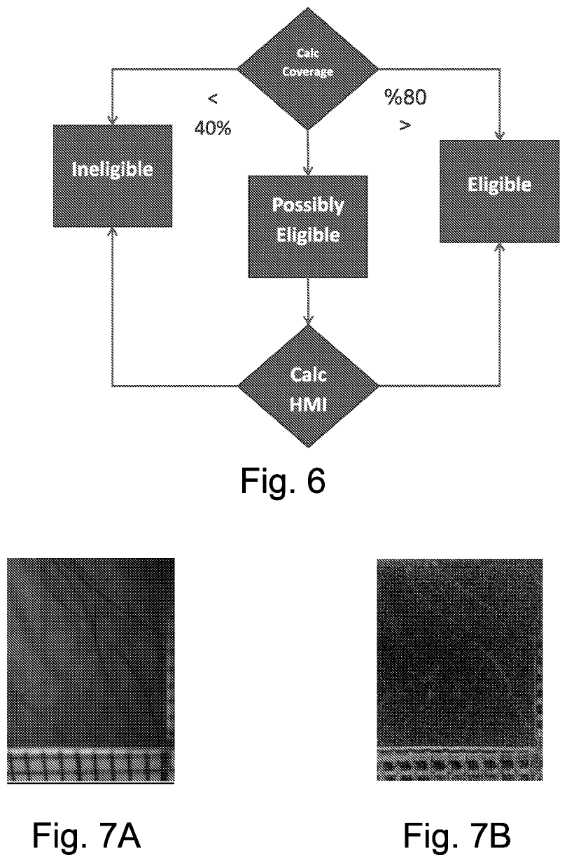

FIG. 6 is a schematic flowchart of the process of calculating the Eligibility of each candidate. At the first step the candidate acquires images or video footages (segments) of his head using an image-capturing device, as described. At the next step, the relative coverage of hair on his head is calculated. If the candidate's head is unshaved, Algorithm 3 described above is applied.

If the candidate's head is shaved, the following steps are performed:
Step 1—divide the number of hairs identified by a known total average number of hair/follicles in adults [9]
Step 2—Determine eligibility if the coverage is above a configured “acceptance threshold” (e.g., above 80%) or below a configured “rejection threshold” (e.g., below 40%).
Step 3—The candidate takes a close-up image of the scalp with a millimetric layout scale attached to the scalp, possibly with the aid of an optical magnifying device.
Step 4—Calculate advanced parameters using Algorithm 4 below, to determine whether they fit within a clinic's desired parameters, such as hair width, distribution, and Hair Mass Index (HMI—an accurate measurement that allows you to calculate the amount of hair (volume, density) and thickness (diameter) on certain areas of the scalp).

### Analysis Routine 8—Calibration

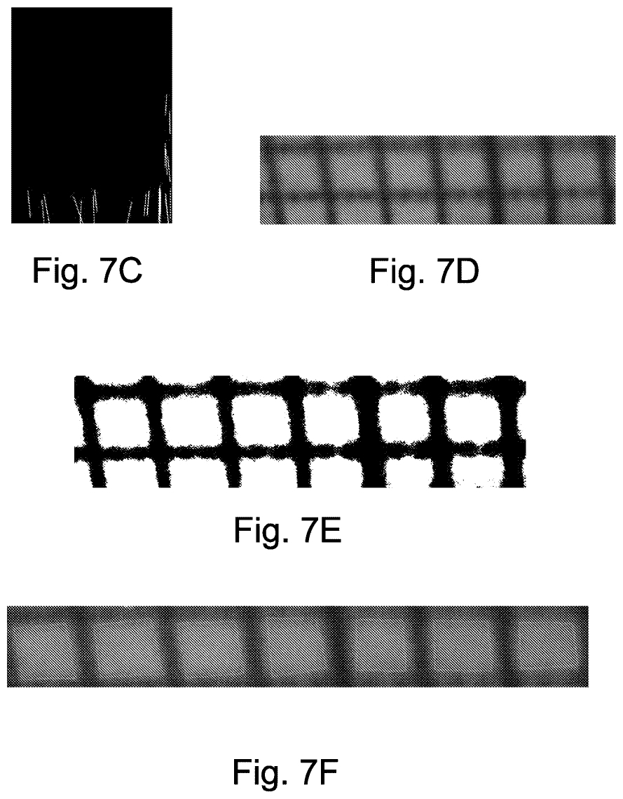

Input: Image frame of unshaved scalp close-up with a millimetric layout attached to the scalp, with a known square dimension, as shown in FIG. 7A.
Output: Pixel size in this image frame

### Exemplar Implementation:

### Step 1—Crop the Area Showing the Millimetric Layout:

1) Covert the image to grayscale
2) Calculate the gradient of the image [8] intensity (horizontal change and vertical change in pixel intensity).
3) Classify pixels using thresholding on the gradient results and blurring/smoothing (average over a window), as shown in FIG. 7B.
4) Perform the Hough line transform (a transform used to detect straight lines) to detect vertical lines, as shown in FIG. 7C.
5) Collect all the line's heights and take the median value as the height of the slice, as shown in FIG. 7D.
6) Slice a rectangle above and to the right of the identified lines with padding gives a skull segment that will be used for the other analysis.

### Step 2—Calibrate the Pixel Dimensions:

1) Convert the image to grayscale
2) Classify pixels to black lines vs white background using dynamic thresholding, as shown in FIG. 7E.
3) Fit squares into the white areas into and locate the square vertexes, as shown in FIG. 7F. The square fitting is achieved by using gradient search to locate the polynomic contours and fit a square for every contour.
4) By ordering each square's vertexes counter-clockwise, to find horizontal and vertical angles for each edge, as shown in FIG. 7G.
5) Get the mean value of angles gives the rotation angle of the millimetric layout in the image. Use the rotation angle to rotate the image so that the vertical lines form 90° with the y-axis, as shown in FIG. 7H.
6) Search along the horizontal axis and find the maximal gap for each row. Gaps above a certain threshold are taken as a square edge. Take the mean value of the collection of edges as the edge value, and estimate the ratio between with edge to full edge by finding the square root of the ratio between the black pixels to the white pixels in the sliced image. Calculate pixel length in mm we use the following formula:

$$Pixel\_length = ratio\_white \_to \_full \_edge / mean\_edge \_length$$

### Analysis Routine 9—Hair Width Calculation

Input: Image frame of unshaved scalp close-up with a millimetric layout attached to the scalp, with a known square dimension (FIG. 7A)
Output: Average hair width

### Exemplar Implementation:

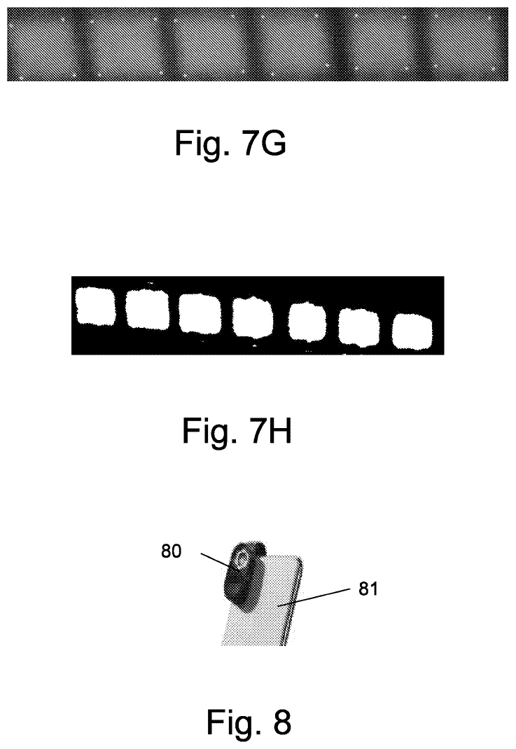

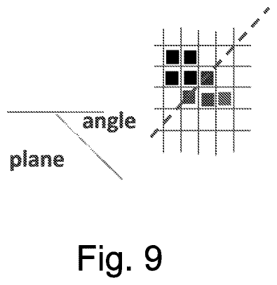

Step 1—Obtain an image close-up using the original device and a commercial optical magnifier (such as a lens) installed on the camera device, or any other way to increase the resolution. FIG. 8 shows a magnifier 80 attached to the candidate's smartphone 81.
Step2—Identify hair in the image using Analysis Routine 3.
Step3—Using the angle of each image from Step 2, obtain the average number of pixels making up its width along each hair, as shown in FIG. 9.
Step 4—Perform calibration using Analysis Routine 8 and calculate pixel_length
Step 5—Obtain hair width by multiplying pixel_length×width pixels. This can be obtained per hair, or on average.

### Hair Cover Percentage Estimation:

The hair cover percentage is estimated using a counting number of pixels classified as hair, divided by the total number of pixels in the cropped image.

Other methods for estimating the percentage of hair on the head are possible. Furthermore, other characteristics beyond the percentage of hair, such as color variation (grey hair, etc.), and the attributes identified on a shaved head, can be calculated.

Finally, the estimated head coverage is compared to a predetermined threshold to determine the candidate's eligibility for a hair restoration procedure, namely whether there is sufficient hair to be harvested from hair-covered sections of the head frame to sufficiently improve the hair coverage in the hairless area of the head frame. The sufficient hair coverage level is to be determined by physical and esthetic considerations taken by surgeon 3 and candidate 2.

For example, a 40% hair coverage may be determined as a threshold, below which a candidate is ineligible for hair restoration, while a 80% hair coverage may be determined as a threshold, above which a candidate is eligible for hair restoration, where hair coverage in the mid-range of 40%-80% may require further analysis to be performed. For instance, a candidate analyzed with a mid-range hair coverage may be requested by surgeon 3 to photograph his head with a common magnifying element attached to their smartphone camera, and with a millimeter ruler as a reference, by which a magnified image showing a typical hair density and hair thickness, which may support the candidate's eligibility.

Further estimation tools may be added to consider the density of hair in haired sections of the head, in order to differentiate head areas covered by thin and possibly weak hairs, from head areas covered by sufficiently dense and strong hairs. For example, by further analyzing the RGB coordinates of hair-covered areas.

Performing an instantaneous remote analysis of a hair restoration candidate, based on predetermined clinic parameters, allows the patient as well as the hair restoration clinic to obtain prompt conclusions as to the candidate's eligibility for the procedure, without requiring a physical visit at the clinic.

Although embodiments of the invention have been described by way of illustration, it will be understood that the invention may be carried out with many variations, modifications, and adaptations, without exceeding the scope of the claims. For example, according to an embodiment of the present invention, rather than a hair restoration candidate's head, the remote hair analysis is applied to other parts of a living or non-living object of interest, for analyzing hair or other health or biological information.

Other data can be used beyond optical images, such as LIDAR info, to help map the spatial location of hair on the head. This may help create a 3D model of the head and hair, and help produce a simulated result of the hair restoration procedure.

### Algorithm 4—Advanced Parameter Calculation

Input: Image frame of unshaved scalp close-up with a millimetric layout attached to the scalp, with a known square dimension.
Output: Per-hair and average advanced calculations.

### Exemplar Implementation:

Step 1—Identify hair follicles, and calculate hair width and pixel size using Analysis Routine 9
Step 2—Calculate the HMI:
1) Obtain the size of the skull surface in question using the size of the scalp in pixels, and the calibration parameters from Step 1.
2) Obtain the hair coverage by Sum (hair_lenght*hair_width*number_of_folicle(i)), i=1, . . . , N, when N is the number of hairs.
3) Calculate HMI using: HMI=ceil(S_hair [mm\hat{ }2]/S_skull [cm\hat{ }2])*100.

The above examples and description have of course been provided only for the purpose of illustration, and are not intended to limit the invention in any way. As will be appreciated by the skilled person, the invention can be carried out in a great variety of ways, employing more than one technique from those described above, all without exceeding the scope of the invention.

### REFERENCES

[1] https://en.wikipedia.org/wiki/Grayscale
[2] https://wiki.robojackets.org/Adaptive Thresholding
[3] Ortiz, Joseph & Avouris, Dulci & Schiller, S. & Luvall, Jeffrey & Lekki, John & Tokars, Roger & Anderson, Robert & Shuchman, Robert & Sayers, Michael & Becker, Richard. (2017). Intercomparison of Approaches to the Empirical Line Method for Vicarious Hyperspectral Reflectance Calibration. Frontiers in Marine Science. 4. 296. 10.3389/fmars.2017.00296.
[4] Jonathan Long, Evan Shelhamer, Trevor Darrell. “Fully Convolutional Networks for Semantic Segmentation”, arXiv:1605.06211v1 [cs.CV] 20 May 2016.
[5] https://en.wikipedia.org/wiki/GrabCut
[6] https://en.wikipedia.org/wiki/Opening (morphology)
[7] https://en.wikipedia.org/wiki/Gaussian blur
[8] Image gradient: https://en.wikipedia.org/wiki/Image gradient
[9] Number of hair/follicles on adults: https://www.webmd.com/skin-problems-and-treatments/hair-loss/science-hair

1. A system for performing a remote hair analysis of the head of a hair restoration candidate, comprising:
a) one or more image-capturing devices adapted to connect directly, or through a computer, to the internet, for capturing images or video segments of the head of said hair restoration candidate to be processed, and for transmitting the captured images or video segments to one or more computing devices; and
b) one or more central computing devices, configured with suitable hardware for running an operating software, which is configured to receive images or video segments of hair restoration candidates' heads, and to process and analyze perceptible hair parameters therein for inferring as to the candidates' eligibility for a hair restoration procedure.

2. A system according to claim 1, wherein the one or more central computing devices are configured to receive and analyze consecutive images or video segments of a candidate's head, and to identify one or more anchoring follicles within two or more consecutive images or video frames.

3. A system according to claim 1, wherein the one or more image-capturing devices are adapted to perform a partial or complete analysis of the captured images or video segments and wherein the images or video segments are captured by manually moving the image-capturing devices by the candidate or by another assisting person.

4. (canceled)

5. A system according to claim 1, wherein the image-capturing device is selected from the group of:
a smartphone;
a tablet;
a digital camera; and
Light Detection and Ranging (LIDAR) sensors.

6. (canceled)

7. (canceled)

8. A method for performing a remote hair analysis of the head of a hair restoration candidate, comprising:
a) capturing, by one or more image-capturing devices adapted to connect directly, or through a computer, to the internet, images or video segments of the head of said hair restoration candidate to be processed;
b) transmitting the captured images or video segments to one or more central computing devices;
c) receiving images or video segments of hair restoration candidates' heads; and
d) processing and analyzing perceptible hair parameters, for inferring as to the candidates' eligibility for a hair restoration procedure.

9. A method according to claim 8, wherein the analysis comprises one of the following operations:
classification of pixels as related to hair within a received image, or a single frame of a head scan video of the candidates head, performed by:
a) receiving an input image frame of a shaved head as R, G, B pixels; and
b) classifying pixels that can be associated with human hair follicles
the identification of individual hair follicles formed by groups of residing pixels identified as hair by:
mapping Pixels grid with “hair” and “scalp” and labeling classification results; and
grouping pixel labels to identify full hair;
the identification of hair follicles and the number of hairs within each hair follicle by:
receiving hair classification and one hair follicle with its bounding box; and
grouping pixel labels to identify full hair and detect the number of hair within said hair follicle;
the generation of a profile of each identified hair follicle by:
receiving each Hair follicle represented by a predetermined number of pixels; and
characterizing each hair follicle according to its orientation and the number of hairs within said follicle;
-receiving video segments of a shaved head; and
identifying and characterizing the hair follicles on each scanned segment of said shaved head;
receiving video segments of a shaved head; and
identifying and characterizing the hair follicles on each scanned segment of said shaved head;
the identification and characterization of hair follicles on unshaved head by:
cropping the raw head image to exclude objects and background and to leave only the desired head section; and
classifying pixels that can be associated with a cropped human head.
receiving frames of video segments of a cropped head;
analyzing said frames and identifying pixels related to hair and other pixels, related to the scalp; and
classifying pixels as related to hair or said scalp;
calculating the estimated hair coverage by:
receiving frames of video segments and cropping the head from each frame by:
creating a Semantic Segmentation Mask;
creating a Grabcuts Mask;
joining said masks to create a final cropping mask;
removing imperfections from said final cropping mask using morphological closing and opening;
classifying each pixel as scalp or hair by:
creating a Color Neighboring Classification;
creating a Luminosity and Saturation Classification;
combining said classifications into a final score;
determining a threshold for classifying pixels having a score below said threshold as related to scalp and pixels having a score above said threshold as related to hair;
calculating the final hair coverage percentage by:
applying said threshold on the final score image and calculating the percentage of the above-threshold score pixels being related to hair, with respect to the below-score pixels being related to skin;
counting the number of pixels classified as hair and dividing the counted number by the total number of pixels in the cropped image.

10. A method according to claim 9, comprising:
a) converting the input image to grayscale;
b) identifying pixels which can be associated with hair; and
c) combining the classification results from the preceding step, based on logical functions or weighted sums, or others;
d) identifying full hair by linking the pixels identified as hair according to the previous section into groups, based on full adjacency; and
e) for each frame, estimating the number of follicles and their location;
f) for every bounding box, creating an orthogonal line that goes through said bounding box;
g) scanning pixels and counting the number of times they flip from H to S and vice versa in each classified image; and
h) Returning the maximal count from the preceding step.

11. (canceled)

12. (canceled)

13. (canceled)

14. (canceled)

15. (canceled)

16. A method according to claim 8, further comprising analyzing a series of
consecutive images, or a video clip as a series of consecutive frames, where anchoring follicles identified within two or more consecutive frames, are used as references to calculate the displacement of the anchor follicles between the consecutive frames.

17. (canceled)

18. A method according to claim 9, comprising:
a) for each frame, identifying and characterizing hair follicles in said frame and in its subsequent frame;
b) Finding the intersection group of follicles, to avoid excessive counting; and
c) Identifying follicles in the intersection group by estimating, for every follicle found in a frame, its location in the subsequent frame,
wherein follicles without matches in consecutive frames are assumed to be new, and are added up to the general follicles count.

19. (canceled)

20. A method according to claim 2, wherein the classification is done by using a classifier based on the Convolutional Neural Network model, which was trained using deep learning, to identify objects on an image and segment the image into different classes, or by applying grabcuts algorithm for image segmentation, based on graph cuts with oval approximation.

21. (canceled)

22. (canceled)

23. (canceled)

24. A method according to claim 9, comprising determining the change in color
of every pixel, compared to its neighborhood by:
a) applying Gaussian blur on the color image in an RGB color plane;
b) creating a new image of the distance from the original to the blurred image to, find localized changes;
c) applying a second Gaussian blur to the new image to find areas with substantial change; and
d) classifying pixels with rapid changes with respect to their neighboring pixels as hairs.

25. A method according to claim 9, comprising analyzing the luminosity and
saturation of the images in the HLS color plane by:
a) converting the image from RGB to HLS color plane;
b) estimating the geometric mean of L and S components for the neighborhood of every pixel;
c) applying Mean Threshold to estimate the differences in lighting for each neighborhood;
d) removing outliers according to a predetermined threshold; and
e) scaling inliers to scale from “probably hair” (dark) to “probably skin” (light).

26. (canceled)

27. (canceled)

28. A system for performing a remote hair analysis of the head of hair restoration candidate, comprising:
a) one or more image capturing devices adapted to connect directly, or through a computer, to the internet, for capturing images or video segments of the head of said hair restoration candidate to be processed, and for transmitting the captured images or video segments to one or more central computing devices; and
b) one or more central computing devices, configured with suitable hardware for running an operating software, which is configured to receive images or video segments of hair restoration candidates heads, and to process and analyze perceptible hair parameters therein for inferring as to the candidates' eligibility for a hair restoration procedure.

29. A system according to claim 28, wherein the capturing device is selected from
the group of:
a smartphone;
a tablet; and
a digital camera;

30. A system according to claim 28, wherein the candidate's head is shaved or unshaved head.

31. A system according to claim 28, wherein the one or more central computing devices are configured to:
receive and analyze images a candidate's head, and to identify one or more follicles, and for each, one or more property like angle or number of hair; and
receive and analyze consecutive images or video of a candidate's head, and to identify a total collection of follicles and their hair on the candidate's head.

32. (canceled)

33. A system according to claim 28, wherein the one or more central computing devices are configured to receive and analyze consecutive images or video of a candidate's head, and to identify one or more anchoring follicles within two or more consecutive images or video frames.

34. (canceled)

35. A system according to claim 28, wherein a total coverage in unshaved head is calculated based on:
identifying the portion of the image that belongs to the head, classifying pixels that belong to the scalp versus the hair, and calculating the percentage of hair coverage accordingly, or
the total number of follicles or hair identified, and the total number of expected follicles or hairs in adults.

36. A system according to claim 28, wherein a calibration method is attaching a marked layout with known distances on the head at the time of taking the pictures, or is used to determine the equivalent value of each pixel in the image.

37. (canceled)

38. (canceled)

39. (canceled)

40. (canceled)

41. (canceled)

42. A system according to claim 28, wherein a magnification device is used in conjunction with the capturing device, in order to improve the resolution and clarity.
